(19) United States
(12) Patent Application Publication
(10) Pub. No.: US 2011/0054628 A1
(43) Pub. Date: Mar. 3, 2011

(54) REFLEX FIXATION GEOMETRY REVISION AND RECONSTRUCTION SYSTEM REVERSE ARTICULATION

(76) Inventors: Stephen A. Banks, Baulkham Hills (AU); Michael J. Ribot De-Bressac, Baulkham Hills (AU); Timothy McTighe, Chagrin Falls, OH (US)

(21) Appl. No.: 12/548,358

(22) Filed: Aug. 26, 2009

(51) Int. Cl. A61F 2/34

(52) U.S. Cl. 623/22.19; 623/22.35

(57) ABSTRACT

An orthopedic device is disclosed for restoring the normal or natural joint mechanics in, for example, a hip joint. The device includes a first component, such as an acetabular component in a hip implant, that includes a convex articulation surface and a second component, such as a femoral component in a hip implant, that includes a concave articulation surface. It will be appreciated that the convex articulation surface of the device disclosed herein articulates with the concave articulation surface in a mating engagement that may be reversed with respect to the traditional hip implant, in which the concave articulation surface is part of the acetabular component and the convex articulation surface is part of the femoral component.

### CROSS-REFERENCE TO RELATED APPLICATIONS

Not Applicable.

### STATEMENT REGARDING FEDERALLY SPONSORED RESEARCH OR DEVELOPMENT

Not Applicable.

### BACKGROUND

1. The Field of the Invention.

The present disclosure relates generally to the field of artificial joints and joint implants, and more particularly, but not necessarily entirely, to acetabular reconstruction using an artificial hip prosthesis having an articulation that is reversed with respect to the normal hip prosthesis.

2. Description of Related Art

It is common practice in the orthopedic industry to use artificial implants to replace diseased, damaged or otherwise compromised joints, such as in the hip, knee, shoulder, or spine. For example, the human hip joint is formed by the acetabulum of the pelvis on one side and the proximal femur on the other. The hip joint acts mechanically as a ball and socket joint, wherein the ball-shaped head of the proximal femur is positioned within the socket-shaped acetabulum of the pelvis. In a total hip arthroplasty or joint replacement, both the femoral head and the surface of the acetabulum are replaced with prosthetic devices. A total hip replacement is typically used when both the natural femoral head and acetabulum are diseased or damaged.

A traditional artificial hip implant includes an acetabular component and a femoral component. An acetabular shell or cup component, which is traditionally hemispherical in shape and attachable to the acetabulum, may be attached to the acetabulum in a cemented application or in a cementless application, i.e., a press-fit with osseous growth fixing the shell to the bone. A bearing or liner may be secured within a cavity of the acetabular shell using several different mechanical locks to secure the bearing or liner to the shell component.

On the femoral side, a traditional femoral implant may be located in the medullary canal of the femur and commonly has a spherical head and an elongated stem. The spherical head may be seated in and articulate with the acetabular bearing or liner.

When the artificial hip implant itself becomes damaged or the bone surrounding or contacting the hip implant becomes further diseased or damaged, it is sometimes necessary for a surgeon to repair the prosthetic hip joint using a reconstruction or revision hip implant. Currently in a reconstruction hip surgery, surgeons may place a reconstructive cage or shell into a deficient acetabulum on the pelvis side of the hip joint along with a bone graft (either allograft or a substitute) into which the cemented cup for receiving a prosthetic femoral head is placed in the best position possible to obtain a more stable joint. Cementing the cup into the cage, independent of the position of the cage, allows for optimum angulation of the cup for increased stability.

Bone loss at or around the acetabulum often results in the cage being attached to the acetabulum in a position that is more vertical than desired. In other words, a base of the cage may be rotated vertically with respect to a vertical midline of the patient. The result of the vertical placement of the cage leads to an undesirable placement of the cup.

There are some known designs that are indicated for use as a cementless device and allow a certain, but often inadequate, flexibility in terms of orientation of the inner liner to provide the best stability. Many of these devices are somewhat ‘eccentric’ or off axis and do not have adequate initial fixation or provision for secondary biological or bone fixation.

The goal of an artificial implant is to restore the natural biomechanics of the natural joint. However, restoring the biomechanics of the joint continues to be a difficult problem. It is noteworthy that none of the devices known to applicant provides a hip implant that satisfactorily restores the mechanics of the hip joint. There is a long felt, but currently unmet, need for a hip implant that satisfactorily restores the natural joint mechanics of the hip joint, as illustrated by the number of hip implants in the marketplace attempting to restore the natural joint mechanics.

Despite the advantages of many of the known devices, improvements are still being sought. However, these known devices are also characterized by several disadvantages that may be addressed by the present disclosure. The present disclosure minimizes, and in some aspects eliminates, the failures, and other problems, of these devices by utilizing the methods and structural features described herein.

The features and advantages of the disclosure will be set forth in the description which follows, and in part will be apparent from the description, or may be learned by the practice of the disclosure without undue experimentation. The features and advantages of the disclosure may be realized and obtained by means of the instruments and combinations particularly pointed out in the appended claims.

### BRIEF DESCRIPTION OF THE DRAWINGS

The features and advantages of the disclosure will become apparent from a consideration of the subsequent detailed description presented in connection with the accompanying drawings in which:

FIG. 1 is a schematic view of an orthopedic device, specifically an embodiment in the form of a hip implant, including a first component and a second component made in accordance with the principles of the present disclosure;

FIG. 1A is a side view of a part of the first component of FIG. 1 and illustrating a convex articulation surface;

FIG. 2 is an exploded side, perspective view of an embodiment of a first component of the orthopedic device made in accordance with the principles of the present disclosure;

FIG. 3 is a side, perspective view of an another embodiment of the first component of the orthopedic device made in accordance with the principles of the present disclosure;

FIG. 4 is a bottom perspective view of the embodiment of the first component of the orthopedic device of FIG. 3;

FIG. 5 is a bottom view of the embodiment of the first component of the orthopedic device of FIG. 3;

FIG. 6 is a schematic side view of an embodiment of the second component of the orthopedic device made in accordance with the principles of the present disclosure;

FIG. 7 is a schematic side view of another embodiment of the second component of the orthopedic device made in accordance with the principles of the present disclosure;

FIG. 8 is a schematic side view of another embodiment of the second component of the orthopedic device made in accordance with the principles of the present disclosure;

FIG. 9 is a schematic side view of another embodiment of the second component of the orthopedic device made in accordance with the principles of the present disclosure;

FIG. 10 is a schematic side view of another embodiment of the second component of the orthopedic device made in accordance with the principles of the present disclosure;

FIG. 10A is a schematic top view of a stem component of the second component of the orthopedic device of FIG. 10 and made in accordance with the principles of the present disclosure;

FIG. 10B is a schematic bottom view of a proximal body portion of the second component of the orthopedic device of FIG. 10 and made in accordance with the principles of the present disclosure;

FIG. 10C is a schematic bottom view of a concave articulation surface portion of the orthopedic device of FIG. 10 and made in accordance with the principles of the present disclosure;

FIG. 10D is a schematic top view of the concave articulation surface portion of the orthopedic device of FIG. 10 and made in accordance with the principles of the present disclosure;

FIG. 11 is a schematic side, cross-sectional view of an embodiment of the orthopedic device made in accordance with the principles of the present disclosure;

FIG. 12 is a schematic side, cross-sectional view of an embodiment of the orthopedic device utilized in a revision surgery and made in accordance with the principles of the present disclosure; and

FIG. 13 is a schematic side, cross-sectional view of another embodiment of the orthopedic device utilized in a revision surgery and made in accordance with the principles of the present disclosure.

### DETAILED DESCRIPTION

For the purposes of promoting an understanding of the principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the disclosure claimed.

Before the present orthopedic device and method of restoring joint mechanics in a joint, such as a hip joint, are disclosed and described, it is to be understood that this disclosure is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present disclosure will be limited only by the appended claims and equivalents thereof.

It must be noted that, as used in this specification and the appended claims, the singular forms “a,” “an,” and “the” include plural referents unless the context clearly dictates otherwise.

In describing and claiming the present disclosure, the following terminology will be used in accordance with the definitions set out below.

As used herein, the terms “comprising,” “including,” “containing,” “characterized by,” and grammatical equivalents thereof are inclusive or open-ended terms that do not exclude additional, unrecited elements or method steps.

As used herein, the phrase “consisting of” and grammatical equivalents thereof exclude any element, step, or material not specified in the claim.

As used herein, the term “proximal” shall refer broadly to the concept of portion nearest to the center of a patient's body, or from the point of origin. For example, a natural femoral bone includes a proximal end having a femoral head that forms part of a hip joint proximally and a distal end having femoral condyles that form part of the knee joint distally. Thus, the proximal femur is so named because it is the proximal-most portion of the femur and is nearest to the center of the patient's body. As another example, a patient's knee is proximal with respect to the patient's toes.

On the other hand, as used herein, the term “distal” shall generally refer to the opposite of proximal, and thus to the concept of a portion farthest from a midline or trunk of a patient's body, depending upon the context. Thus, the distal femur, for example, is so named because it is the distal-most portion of the femur and is farthest from the patient's midline or trunk. As another example, a patient's fingers are distal with respect to the patient's shoulder.

As used herein, the phrase “in an at least a partially proximal-to-distal direction” shall refer generally to a two-dimensional concept of direction in which the “proximal-to-distal” direction defines one direction or dimension. An item that extends in a non-parallel direction with respect to the “proximal-to-distal” direction, that is, at a non-straight angle thereto, thereby involves two components of direction, one of which is in the “proximal-to-distal” direction and the other having some other component of direction, for example a direction orthogonal to the “proximal-to-distal” direction. As a specific example, a patient's natural femur extends in a substantially proximal-to-distal direction.

Referring now to the figures, it will be appreciated that the present disclosure relates to an orthopedic device for replacing a joint in a patient's body. The device 10 of the present disclosure may function to restore the natural joint mechanics, or in other words targets restoration of the natural joint mechanics. With proper joint stability and proper restoration of the joint mechanics, the stress and strains placed on the artificial joint, i.e., the bearing or articulating surfaces at the joint interface, will be reduced, thereby reducing wear debris.

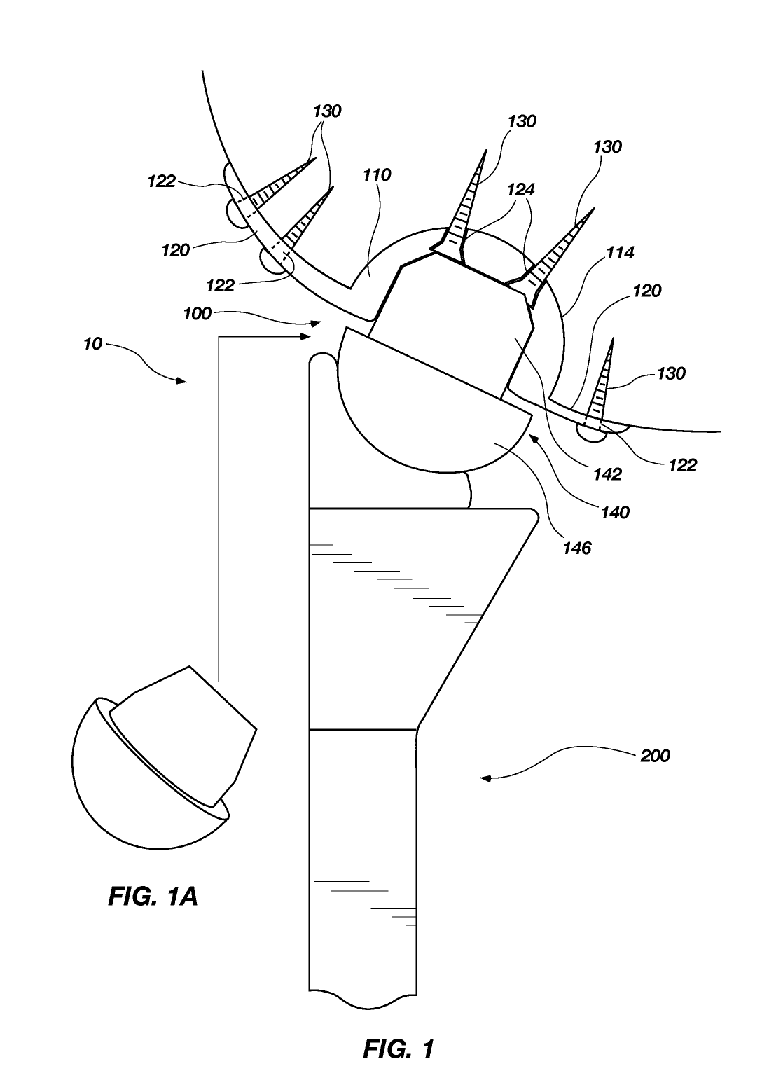

By way of example, FIG. 1 illustrates an embodiment of the orthopedic device 10 of the present disclosure, which may comprise a first component 100 that may be attachable to an acetabulum of a patient and a second component 200 that may be attachable to a proximal femur of the patient.

It will be understood that the first component 100 may comprise a shell 110 that may be directly attached or may be attachable to the acetabulum of the patient. The first component 100 may further comprise an insert 140 that may be attachable to the shell 110.

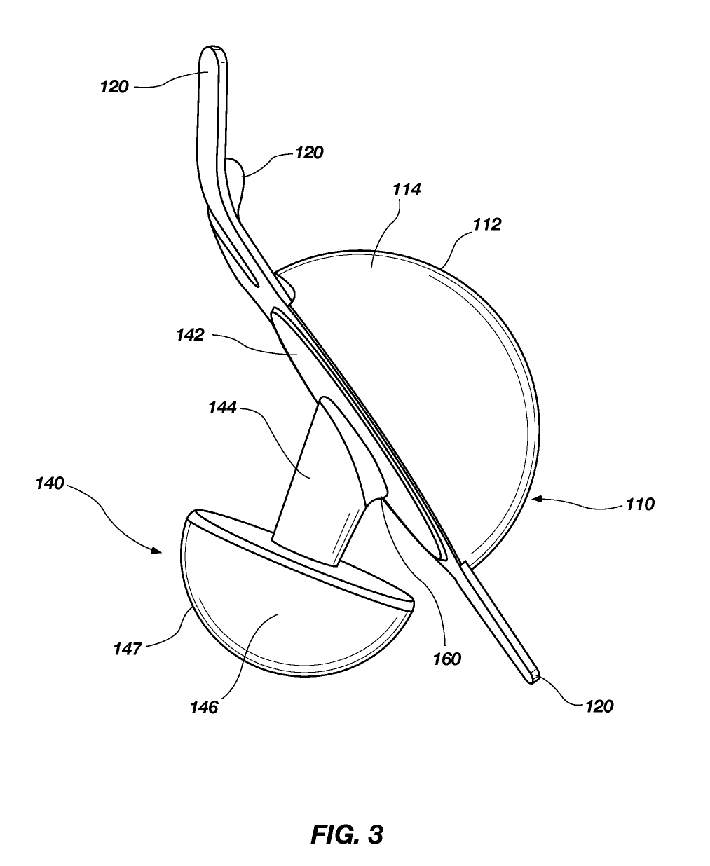

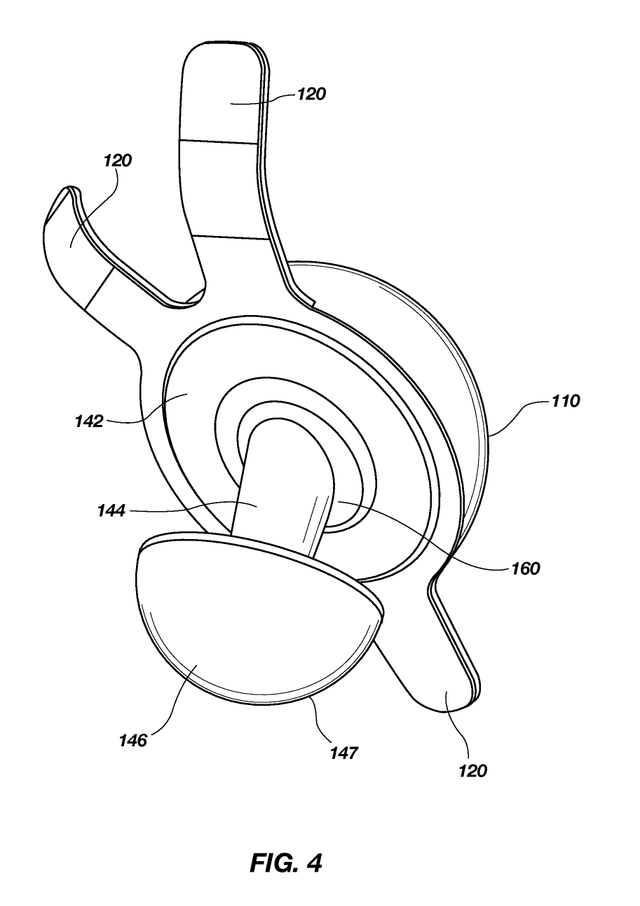

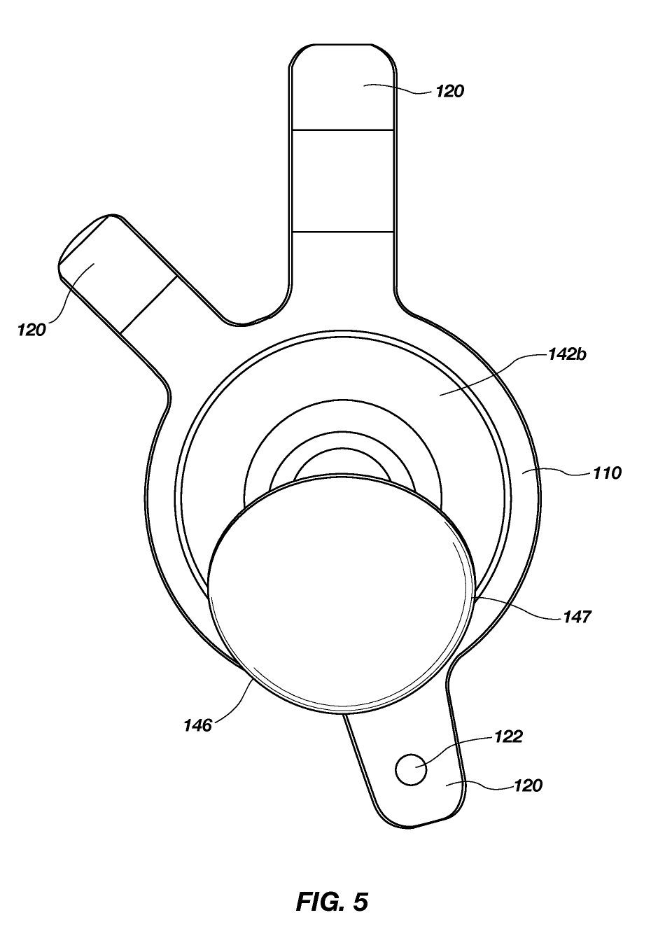

It will be appreciated that the shell 110 may be attached to the acetabulum in any method known, or that may become known in the future, in the art. For example, the shell 110 may be attached to the acetabulum using bone cement or the shell may be attached to the acetabulum via a press-fit between the shell 110 and the bone of the acetabulum itself. As illustrated in FIGS. 1-5, the shell 110 of the first component 100 may include a first body 112 having an outer surface 114 and a cavity 116, which may be defined at least in part by a wall 118. It will be appreciated that the wall 118 may be tapered to form a mating tapered engagement with a portion of the insert 140, which engagement is described more fully below in connection with the insert 140.

The shell 110 may be substantially semi-spherical in shape, but it will be appreciated that other shapes may be utilized without departing from the spirit or scope of the present disclosure. As used herein, the phrase “substantially semi-spherical” means a partial, part or portion of an object that resembles or has some of the characteristics of a sphere, but it should be noted that the above phrase is broad enough to include and does in fact encompass an object having a curved outer surface or an object having a convex surface, whether or not such an object can be characterized as a sphere or portion of a sphere.

As illustrated best in FIGS. 1, and 3-5, at least one flange 120 may extend from the first body 112. It will be appreciated that the type of flange 120 utilized may vary somewhat and may include flanged augments, hooked augments, finned augments and even plain augments, all of which are known in the art. Without regard to the specific flange 120 utilized, the at least one flange 120 may include a first through hole 122 such that a fastener 130, such as a cancellous screw or other fastener, may be extendable into the first through hole 122 and into the surrounding bone. It will be appreciated that each flange 120 may include a first through hole 122 for allowing passage of the fastener 130 therethrough, which may aid in securing the shell 110 to the bone.

Similarly, it will be appreciated that at least one through hole 124, i.e., a second through hole, may be present in the shell 110 itself and the through hole 124 may extend through the wall or tapered wall 118 of the cavity 116 on the inner portion of the shell 110 and may open to the outside of the shell 110 at the outer surface 114 of the first body 112. Thus, a fastener 130 may be extendable into the through hole 124 and into the surrounding bone.

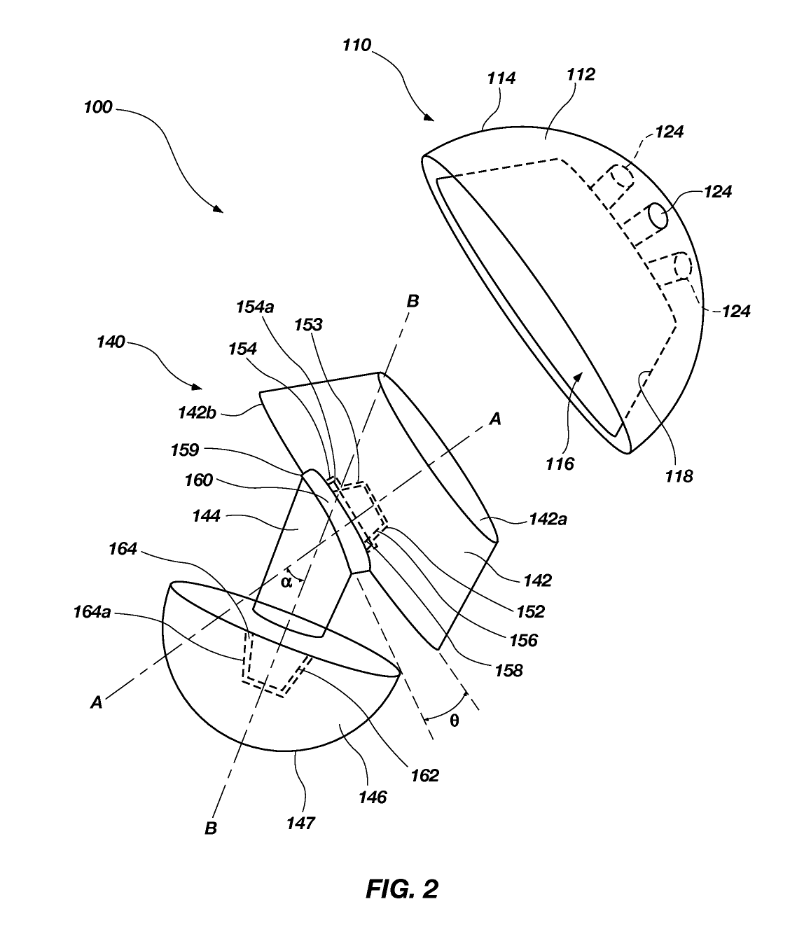

As illustrated in FIGS. 1-5, the insert 140 may have a body 142, a neck 144 and a head 146. It will be appreciated that the body 142 of the insert 140 may be attachable to the cavity 116 of the shell 110. To accomplish the connection, the body 142 of the insert 140 may include a tapered outer surface 150 (illustrated best in FIG. 2), which may structurally correspond with the wall 118 of the shell 110. Thus, the tapered outer surface 150 of the body 142 of the insert 140 may function to matingly engage the wall 118, which may also be tapered, of the shell 110 in a self-locking friction fit. The tapered outer surface 150 of the body 142 of the insert 140 and the tapered wall 118 of the cavity 116 of the shell 110 may each comprise an angle that is within a range of self-locking taper angles.

It will be appreciated that the insert 140 may comprise at least one modular connection with respect to the body 142, the neck 144 and the head 146. In other words, the body 142 may be modular with respect to the neck 144 and the neck 144 may further be modular with respect to the head 146. Thus, the neck 144 may be modular with respect to both the body 142 and the head 146. The at least one modular connection may be a self-locking taper interlock between corresponding components as illustrated, for example, in FIG. 2. It will be appreciated that the insert 140 may be a monoblock piece and does not necessarily have to comprise at least one modular piece. However, it will be appreciated that many advantages may be gained by using a modular design, such as a reduction in inventory, and one of skill in the art can determine when a modular design may be better suited than a monoblock design and vice-versa without departing from the spirit or scope of the present disclosure.

The body 142 may include a top portion 142a and a bottom portion 142b, also referred to herein as a first base portion. Further, a recess 152 may be formed in the bottom or first base portion 142b of the body 142 and may be defined by a tapered sidewall 153. The recess 152 may further be defined by an undulating surface 154 that may be tapered and may open to an exterior portion of the body 142. It will be appreciated that the recess 152 may be present when the body 142 is a modular piece with respect to the neck 144 and may not be present when the body 142 is a unitary, monoblock piece with respect to the neck 144.

The neck 144 of the insert 140 may be affixed or attached to both the head 146 and the body 142. Accordingly, the body 142 and the head may be affixed or attached to the neck 144 in either a modular embodiment or a monoblock embodiment. As illustrated, the neck 144 may extend with respect to the body 142.

As discussed above, if there is a modular connection between the neck 144 and the body 142, then the neck 144 may comprise an outer tapered portion 156 that may engage the corresponding tapered sidewall 153 of the recess 152 of the body 142 in a tapered friction fit. Further, the outer tapered portion 156 of the neck 144 may comprise a plurality of first teeth 158 that may substantially surround a perimeter of the outer tapered portion 156. The undulating surface 154 of the recess 152 in the body 142 may include a plurality of second teeth 154a for engaging the first teeth 158, such that the neck 144 may be indexable in a plurality of differing orientations with respect to the body 142.

A neck 144 that is indexable neck 144 may permit the adjustment of the version angle and the neck shaft angle to provide maximum joint stability. In other words, referring to FIG. 2, it can be seen that the neck 144 may be adjusted to accommodate various varus or valgus angles depending upon the position of the neck 144. For example, by moving and indexing the neck 144 at a six o'clock, nine o'clock or twelve o'clock position, the varus or valgus angle can easily be changed to correspond to a particular patient's anatomy and needs. Further, the neck-shaft angle can be between a range of angles, for example between about a 120° to about a 150° angle, and more specifically between about 125° to about 147° angle. It will be appreciated that the average neck shaft angle may be about a 135° angle.

One embodiment of the insert 140 (illustrated in FIG. 2) may include an imaginary central body axis represented by the line A-A and an imaginary central neck axis represented by the line B-B, wherein the central neck axis B-B is offset at an angle α with respect to the central body axis A-A. It will be appreciated that the angle α between axes may be within a range of angles of about ten degrees to about sixty degrees, and more specifically within a range of about twenty degrees to about fifty degrees, and very specifically about thirty-five degrees. An offset central neck axis B-B may be used in situations where bone loss is severe and the position of the shell 110 is too vertically oriented in the acetabulum, such that a neck having an angle may be required to restore the natural joint mechanics. Further, an offset neck 144 that is also indexable may allow for correction of the version angle, thereby promoting stability and increased range of motion.

The neck 144, as opposed to the body 142, may include a second base portion 159 with the tapered outer portion 156 extending from the second base portion 159 (illustrated best in FIG. 2) for engaging the tapered sidewall 153 of the recess 152 in a friction fit. The neck 144 may further have a support 160 located at the second base portion 159 creating an angular offset of the neck 144 with respect to the first base portion 142a of the body 142 when the neck 144 is attached to the body 142. The angular offset of the neck 144 may thus create differing version angles as the neck 144 may be oriented in each of the plurality of differing orientations that are available from which to choose or select.

It will be appreciated that the support 160 may be shaped as a wedge, to thereby create the angular offset referred to above. In other words, the support 160 may have a triangular cross-section. The angular offset represented by θ and created by the support 160 may be within a range of angles of about three degrees to about twenty degrees, and more specifically within a range of angles of about six degrees to about fifteen degrees.

Likewise, in the modular embodiment with respect to the head 146, the neck 144 may comprise an outer tapered portion 162 and may include a plurality of teeth, similar to the plurality of teeth 158 at the opposite side of the neck 144, for indexing purposes, if desired. Further, in the modular connection between the neck 144 and the head 146, the head 146 may include a recess 164 defined by a tapered sidewall 164a for receiving and matingly engaging the outer tapered portion 162 of the neck 144.

The head 146 of the first component 100 may be shaped and sized in various dimensions. However, it may be advantageous to use an oversized or large head 146 that may marry or matingly engage a corresponding surface, i.e., a concave articulation surface 214 described more fully below, in the second component 200. When an oversized or large head 146 is used, the result may be increased joint stability. Recent trends in the orthopedic industry tend to favor oversized or large heads due to the stability that may be provided thereby.

Whether in a modular embodiment or a monoblock embodiment, the head 146 of the insert 140 may include a convex articulation surface 147. It will be appreciated that with the convex articulation surface 147 being formed as part of the first component 100, the convex articulation surface is thereby located on the acetabular side of the device 10. Thus, the traditional hip implant system, in which the convex articulation surface is located on the femoral side of the hip implant, is altered by the present disclosure.

The first component 100 of the present disclosure may be a monoblock component or it may include at least one modular junction, or a plurality of modular junctions. For example, as illustrated in FIG. 2, any combination of the components could be modular and could create a modular junction. Specifically, the shell 110, the insert 140, the neck 144 and the head 146 of the first component 100 may all be modular pieces to create a quad-body component 100. Additionally, other combinations of the above components may be modular to form a bi-body or tri-body embodiment.

Further, the first component and its various modular parts may be manufactured from various biocompatible materials without departing from the spirit of scope of the present disclosure. For example, the shell 110, neck 144 and head 146 may be manufactured from relatively hard biocompatible materials, including chrome-cobalt, titanium, titanium alloys and other metallic materials, and also ceramic materials or diamond materials. Further, the insert 140 may be manufactured from various biocompatible materials including both soft and hard materials, including polymeric materials, chrome-cobalt, titanium, titanium alloys and other metallic materials, and also ceramic materials or diamond materials. However, it will be appreciated that the type of materials may vary somewhat, such that the insert 140 may be manufactured from a harder biocompatible material and the shell 110, neck 144 and head 146 may be manufactured from a softer biocompatible material without departing from the scope of the present disclosure.

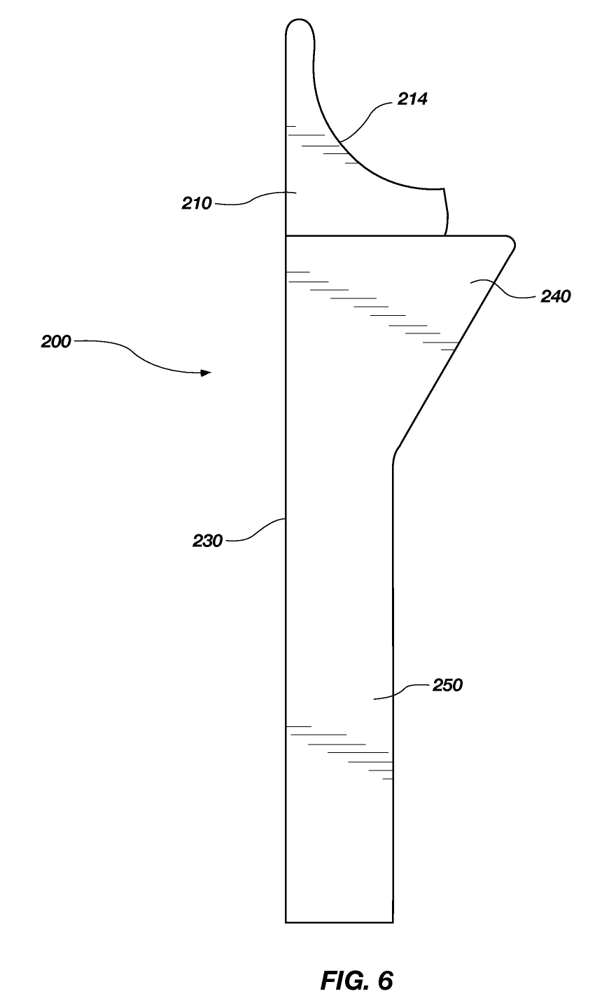

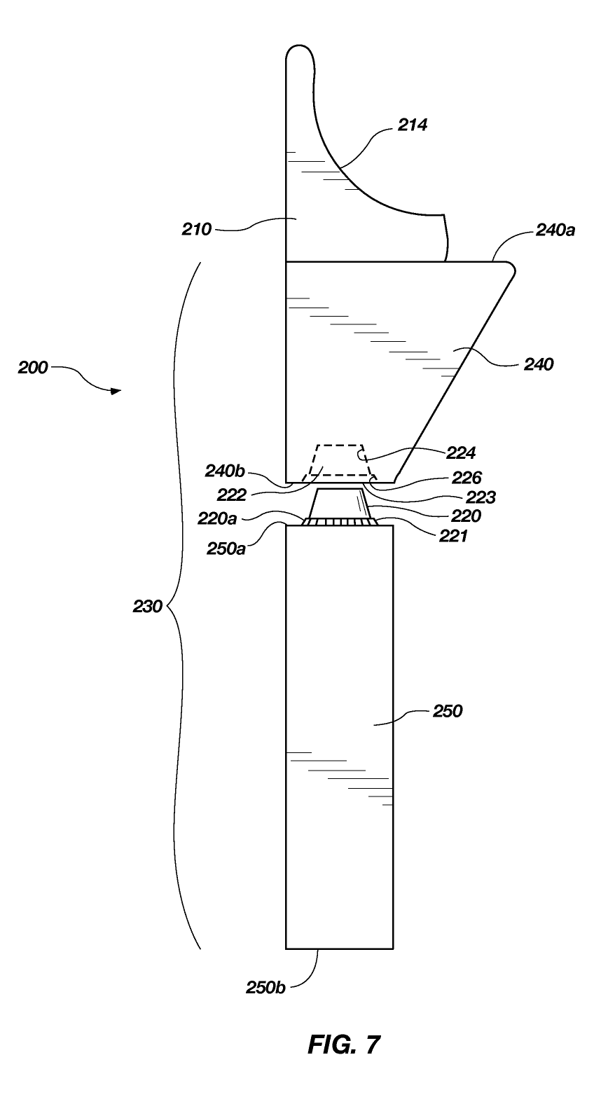

Referring now to FIGS. 6-10B, the second component 200 is illustrated and may comprise a concave articulation surface portion 210 and a femoral stem component 230. It will be appreciated that the concave articulation surface portion 210 may be part of a monoblock stem embodiment (illustrated best in FIG. 6) or it may be part of a modular stem embodiment (illustrated best in FIGS. 7-10) and may comprise a plurality of modular attachment pieces 212 each having a different shape or size.

In either embodiment whether monoblock or modular, the concave articulation surface portion 210 may further comprise a concave articulation surface 214. The concave articulation surface portion 210, including the concave surface 214 itself, may function along with the head 146 of the insert 140 and its convex articulation surface 147 to form an artificial joint. It will be appreciated that the concave articulation surface 214 may be part of the second component 200, which is a femoral component, and the convex articulation surface 147 may be part of the first component 100, which is an acetabular component, and the two surfaces 214 and 147 may engage each other in the formation of the artificial joint.

The concave articulation surface 214 may be a modular attachment piece that may be securable to the concave articulation surface portion 210. In the modular embodiment of the concave articulation surface 214, there may be a plurality of modular attachment pieces that function as concave articulation surfaces 214, and each may have a different thickness than the others. Each of the plurality of modular attachment pieces that function as a concave articulation surface 214 may provide a surgeon with an ability to adjust the joint mechanics of the hip or other joint by utilizing a particular thickness for the concave articulation surface 214. Each of the modular attachment pieces that function as a concave articulation surface 214 may be lockable to the concave articulation surface portion 210 in any modular interlocking mechanism that is known or that may become known in the future in the art. For example, a self-locking tapered fit, i.e., a morse taper may be utilized. However, a morse taper is simply one of a myriad of mechanisms that may be utilized to interlock the modular concave articulation surface 214 to the concave articulation surface portion 210.

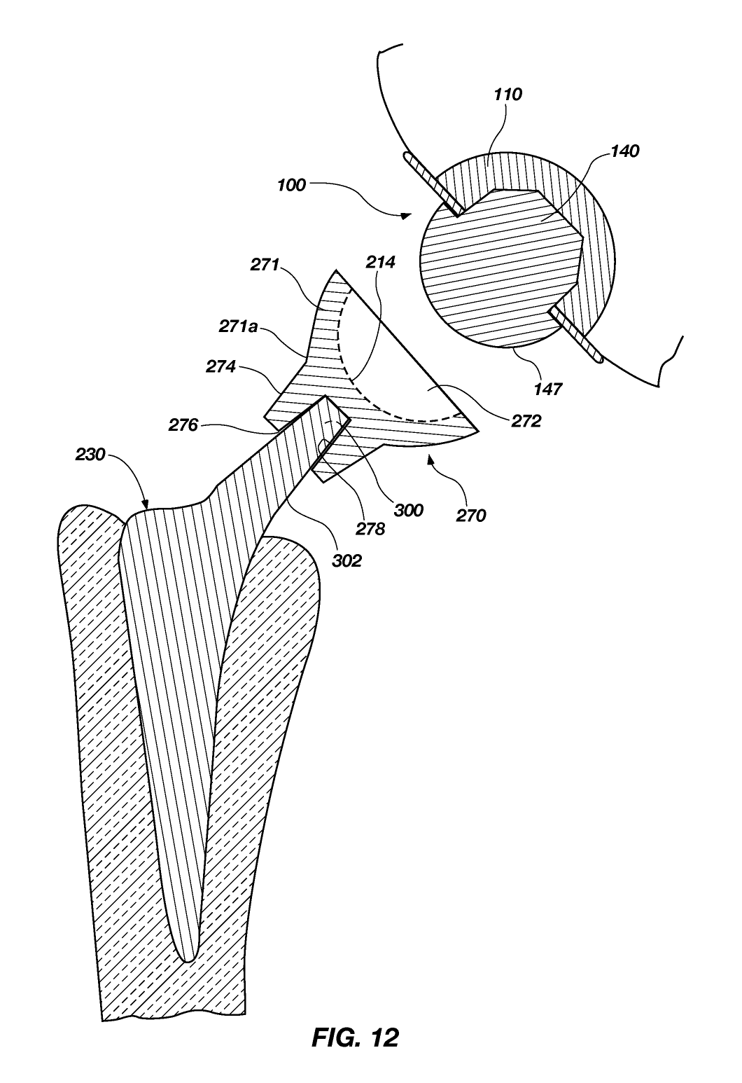

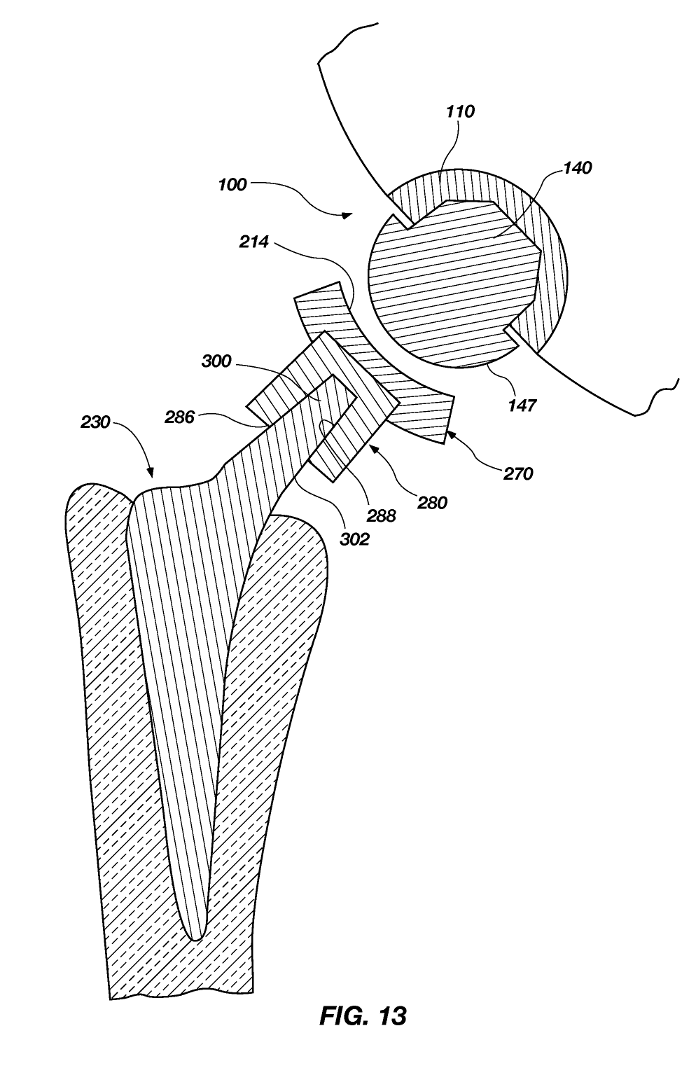

Referring now to FIGS. 12 and 13, two different embodiments of a modular attachment piece 270, which may be utilized in a revision surgery, are illustrated. It will be appreciated that the modular attachment pieces 270 illustrated in FIGS. 12 and 13 may be utilized in connection with the various embodiments disclosed herein for the first component 100, which itself may be attachable to the acetabulum of the patient. Accordingly, the first component 100 may include the shell 110, which may be directly attached to the acetabulum, and the insert 140 that may be attachable to the shell 110 as disclosed herein.

The embodiments of modular attachment pieces 270 of FIGS. 12 and 13 may be configured and dimensioned to attach to the femoral stem component 230 of an artificial hip implant 10. For example, in a revision surgery where the acetabular component 100 of the implant has failed and must be replaced, but where the femoral component 230 is well placed and has good stability in the femur and is not damaged or otherwise compromised, the modular attachment piece 270 of the present disclosure may be utilized to transform a traditional hip implant into a reverse articulation hip implant as disclosed herein.

The modular attachment pieces 270 of FIGS. 12 and 13 may each comprise a concave articulation surface 214, which may engage with the convex articulation surface 147 of the first component 100 to form an artificial joint. Referring specifically to FIG. 12, the modular attachment piece 270 may comprise a cup 271 with a cavity 272 formed therein. The cavity 272 may be substantially semi-spherical and may be defined by the concave articulation surface 214. The modular attachment piece 270 may also comprise a support 274 that may extend downwardly from a base 271a of the cup 271. A recess 276 may be formed within the support 274 and may be defined by a tapered sidewall 278 for matingly engaging a tapered end portion 300 of a neck portion 302 of the femoral component 230 in a friction fit. It will be appreciated that the cup 271 of the modular attachment piece 270 may be configured and dimensioned to either semi-constrain or constrain the convex articulation surface 147. Thus, the modular attachment piece 270 may be directly attachable to the femoral stem component 230 during a revision surgery, which may be existing and implanted within the femur of the patient.

Referring specifically now to FIG. 13, the second component 200 may comprise an adapter 280 that may be attachable to the modular attachment piece 270. It will be appreciated that the modular attachment piece 270 may be attached in any number of ways to the adapter 280, including tapered connections, key and hole connections, bayonet connections, or any other modular connection. It will be appreciated that any modular connection is intended to fall within the scope of the present disclosure. A recess 286 may be formed within the adapter 280 and may be defined by a tapered sidewall 288. The tapered sidewall 288 may matingly engage the tapered end portion 300 of the neck portion 302 of the femoral component 230 in a friction fit. It will be appreciated that there may be a plurality of modular attachment pieces 270 that may be utilized and attached to the adapter 280. Further, as illustrated, the concave articulation surface 214 of the modular attachment piece 270 in FIG. 13 may not substantially constrain the convex articulation surface 147.

It will be appreciated that the concave articulation surface 214, whether modular or monoblock, and perhaps the concave articulation surface portion 210 may be manufactured from any hard biocompatible material. For example, the concave articulation surface 214 and the concave articulation surface portion 210 may both be manufactured from a ceramic, metal, metallic alloys, or even diamond or diamond-based material. However, it will be appreciated that while the concave articulation surface 214, whether modular or monoblock, may be manufactured from any biocompatible, hard material, the concave articulation surface portion 210 may be manufactured from a relatively soft, biocompatible material. For example, the concave articulation surface portion 210 may be manufactured from a polymeric material, which is relatively soft in comparison to ceramic, metal or diamond.

The femoral stem component 230 of the second component 200 may further comprise a proximal body portion 240 and a distal stem portion 250. The femoral stem component 230 may be part of a monoblock stem embodiment (illustrated best in FIG. 6) or it may be part of a modular stem embodiment (illustrated best in FIGS. 7-10).

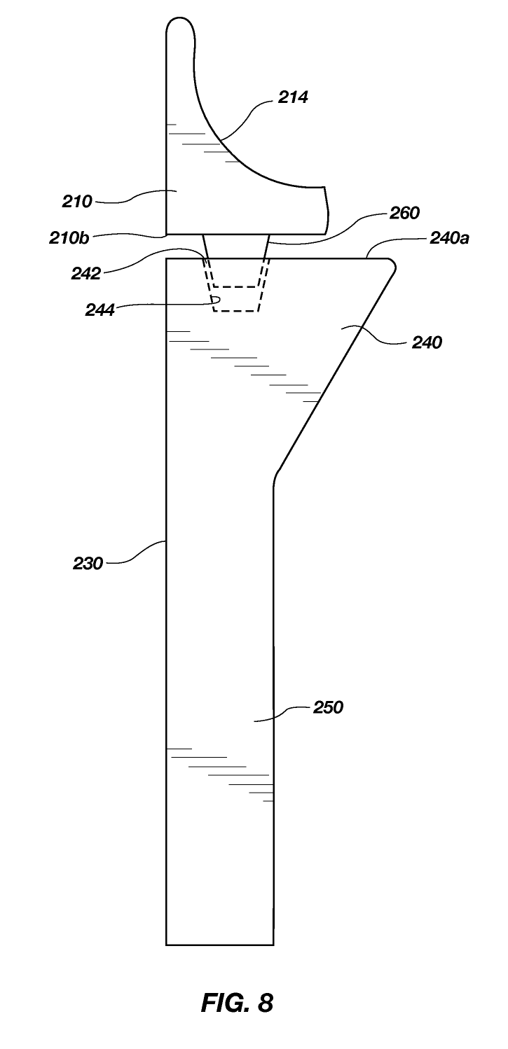

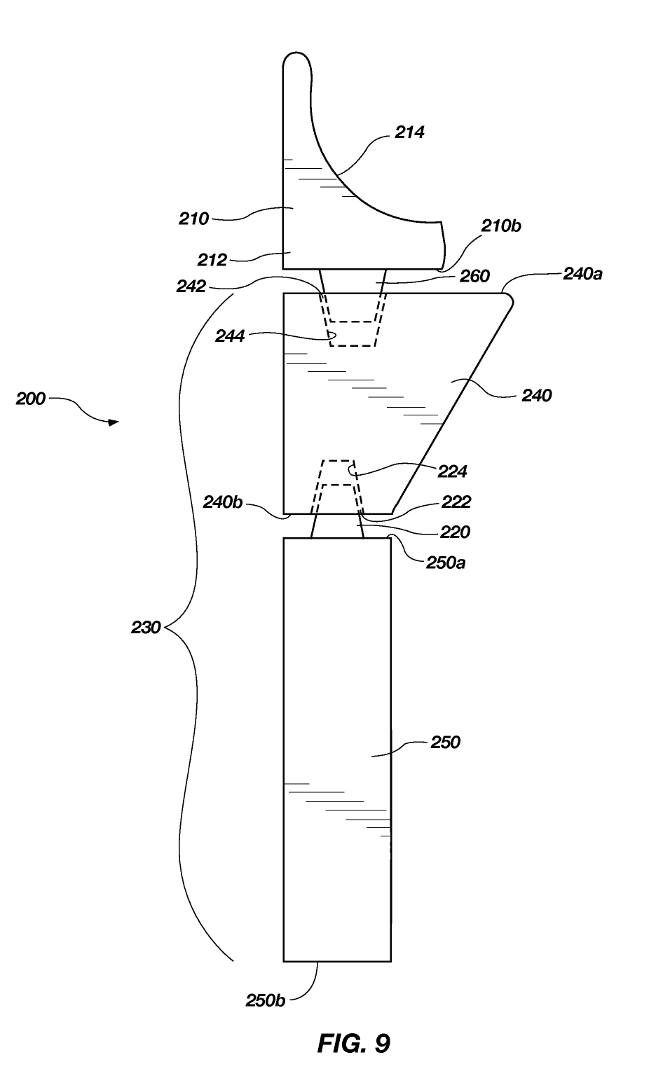

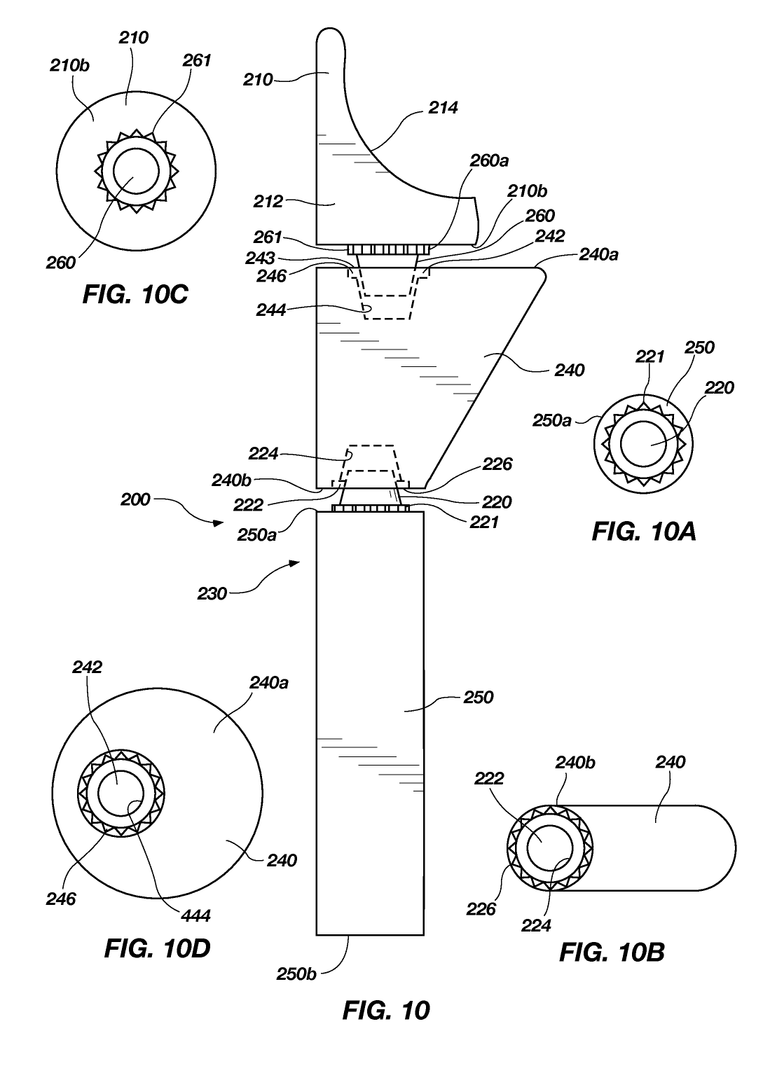

It will be appreciated that the second component 200 may be monoblock stem (illustrated in FIG. 6), a bi-body stem having two modular pieces (illustrated in FIGS. 7 and 8), or a tri-body stem (illustrated in FIGS. 9 and 10).

It will be appreciated that in a monoblock stem embodiment of FIG. 6 the concave articulation surface portion 210 and the femoral stem component 230 may be one, unitary piece. The monoblock stem embodiment may be used with particular type of a patient having predetermined indications that suggest using a monoblock stem.

The modular stem embodiments of FIGS. 7-10 may comprise at least one modular junction, and possibly a plurality of modular junctions, between the modular pieces of the second component 200, for example the concave articulation surface portion 210, the proximal body portion 240 and the distal stem portion 250.

The modularity and potential indexability of the proximal body portion 240, and other modular pieces disclosed herein, may permit a surgeon to index the proximal body portion 240, or other modular piece or pieces, to obtain maximum bone contact. The modularity and indexability of the first and second components 100 and 200 of the present disclosure particularly aid in maximizing bony contact on the medial calcar portion of the femur. In other words, by indexing or moving the proximal body portion 240, or other modular piece, a surgeon can selectably locate the modular piece of the implant within the femur to optimize bony contact and hence implant stability at the bone-implant interface.

Specifically, the bi-body stem embodiment of FIGS. 7 and 8 may comprise one modular junction, which may be formed between the concave articulation surface portion 210, the concave articulation surface 214 itself, the proximal body portion 240 and the distal stem portion 250. More specifically, the modular junction may be formed between: (i) the concave articulation surface 214 and the concave articulation surface portion 210; (ii) the concave articulation surface portion 210 and the proximal body portion 240; or (iii) between the proximal body portion 240 and the distal stem portion 250.

The tri-body stem embodiment of FIGS. 9 and 10, on the other hand, may comprise a plurality of modular junctions between the concave articulation surface portion 210, the concave articulation surface 214 itself, the proximal body portion 240 and the distal stem portion 250, which may be modular pieces with respect to each other. In other words, there may be a modular junction formed between: (i) the concave articulation surface 214 and the concave articulation surface portion 210; (ii) the concave articulation surface portion 210 and the proximal body portion 240; or (iii) the proximal body portion 240 and the distal stem portion 250. It will further be appreciated that any combination of the above modular junctions may be utilized together to form a tri-body stem and without departing from the spirit or scope of the present disclosure.

It will be appreciated that any of the modular junctions described herein, in which at least two modular components or pieces are joined together, may be formed by a first tapered portion 220 or 260 being located on, or formed as part of, one of the modular components or pieces, such as items 210, 214, 240 or 250. The first tapered portion 220 or 260 may engage a tapered sidewall, such as 224 or 244 defining a recess 222 or 242 in another one of the modular components or pieces in a tapered, friction fit, i.e. a morse taper.

While a self-locking friction fit, i.e. a morse tapered friction fit is illustrated and disclosed herein, it should be noted that each of the modular junctions, whether part of the first component 100 or the second component 200, may utilize other modular interlocking mechanisms without departing from the scope of the present disclosure.

It will be appreciated that the modular connection between the first tapered portion 220 or 260 and the tapered sidewall 224 or 244 of the recess 222 or 242 at any modular junction described herein may be as illustrated in FIGS. 7-10. In other words, the modular connection may comprise the tapered, friction fit only as described above, or the modular connection may include additional characteristics such as an indexable feature described below.

More specifically, a modular connection between two modular components or pieces, such as items 210, 214, 240 and 250 may comprise the structure for the tapered, friction fit and may further comprise structure for indexing one modular component with respect to another modular component.

For example, in the embodiment illustrated in FIG. 7 (as well as one of the modular connections in FIGS. 10, 10A and 10B), the modular connection may be between the proximal body portion 240 and the distal stem portion 250. As illustrated in FIGS. 7, 10A and 10B, a plurality of first teeth 221 may be located near the first tapered portion 220, which may be formed on a proximal end 250a of the distal stem portion 250. The plurality of first teeth 221 may surround the entire first tapered portion 220, or alternatively may surround a part or even a majority of the first tapered portion 220 at a base 220a of said first tapered portion 220.

Additionally, a plurality of second teeth 226 corresponding with the plurality of first teeth 221 may be formed as part of the sidewall 224 defining the recess 222 near an opening 223 of said recess 222. In this example, the recess 222 may be formed in a distal end 240b of the proximal body portion 240. Since the plurality of second teeth 226 correspond with the plurality of first teeth 221, the number, shape, size and location of the second teeth 226 may be directly proportional to the first teeth 221. Further, the first teeth 221 may engage the second teeth 226 in a mating engagement and function to allow one modular piece to be indexed with respect to another modular piece. It will be appreciated that as the number of teeth increases the number of predetermined indexable orientations also increases and vice-versa.

In another embodiment illustrated in FIG. 8, the modular connection may be between the concave articulation surface portion 210 and the proximal body portion 240. In this embodiment, only a tapered, friction fit is illustrated, such that the first tapered portion 260 and the recess 242 do not include teeth 221 or 226 for indexing purposes. Further, the first tapered portion 260 may be formed at a distal end 210b of the concave articulation surface portion 210. Further, the recess 242 may be formed in a proximal end 240a of the proximal body portion 240. It will be appreciated that an indexable feature as disclosed herein, such as first and second teeth 221 and 226 that matingly engage each other in an indexable manner, may be utilized in this embodiment to permit the concave articulation surface portion 210 to be indexed with respect to the entire stem component 230.

Referring now to the embodiment in FIG. 9, a tri-body stem is illustrated having two modular connections, which may be as described and disclosed in connection with FIGS. 7 and 8. Even though the indexable feature of the present disclosure is not illustrated in FIG. 9, it will be appreciated that an indexable feature as disclosed herein may be utilized by the present embodiment at one or more of the modular connections without departing from the spirit or scope of the present disclosure.

Referring specifically to FIGS. 10-10D, another tri-body stem embodiment is disclosed in which both modular connections may be tapered, friction fits and may also be indexable. Specifically, the modular connection between the concave articulation surface portion 210 and the proximal body portion 240 may include a plurality of first teeth 261 that may be located near the first tapered portion 260, which may be formed on the distal end 210b of the concave articulation surface portion 210 (see FIG. 10C). The plurality of first teeth 261 may surround the entire first tapered portion 260, or alternatively may surround a part or even majority of the first tapered portion 260 at a base 260a of said first tapered portion 260.

Additionally, a plurality of second teeth 246 corresponding with the plurality of first teeth 261 may be formed in the sidewall 244 of the recess 242 near an opening 243 of said recess 242 (see FIG. 10D). In this example, the recess 242 may be formed in a proximal end 240a of the proximal body portion 240. Since the plurality of second teeth 246 correspond with the plurality of first teeth 261, the number, shape, size and location of the second teeth 246 may be directly proportional to the first teeth 261. Further, the first teeth 261 may engage the second teeth 246 in a mating engagement and function to allow one modular piece to be indexed with respect to another modular piece. It will be appreciated that as the number of teeth increases the number of predetermined indexable orientations also increases and vice-versa. It will be appreciated that the modular connection between the proximal body portion 240 and the distal stem portion 250 as illustrated in FIGS. 10, 10A and 10B may be as described with respect to the modular connection in FIG. 7.

Further, the indexable features of the present disclosure, i.e., the plurality of first teeth 221 or 261 and the plurality of second teeth 226 and 246, may or may not be tapered. If the first teeth 221 and 261 and the second teeth 226 and 246 are tapered, then the result is a double tapered, friction fit. In such an embodiment, a primary taper occurs at the tapered, friction fit between the first tapered portion 220 and 260 and the sidewall 224 and 244 of the recess 222 and 242, while a secondary or back-up taper occurs between the tapered connection of the first teeth 221 and 261 and the second teeth 226 and 246.

Further, it is to be understood that in the various embodiments of a modular connection the first tapered portion 220 may extend from the distal end 250a of the stem component 250, while the first tapered portion 260 may extend from the proximal end 210a of the concave articulation surface portion 210. Further, it will be appreciated that the location of the first tapered portion and the recess may be reversed without departing from the spirit or scope of the present disclosure.

Additionally, each of the various modular connections described and shown herein may be used in various combinations depending upon the surgical or biomechanical need of the patient. Thus, the above modular connections may be mixed and matched without from the spirit or scope of the present disclosure.

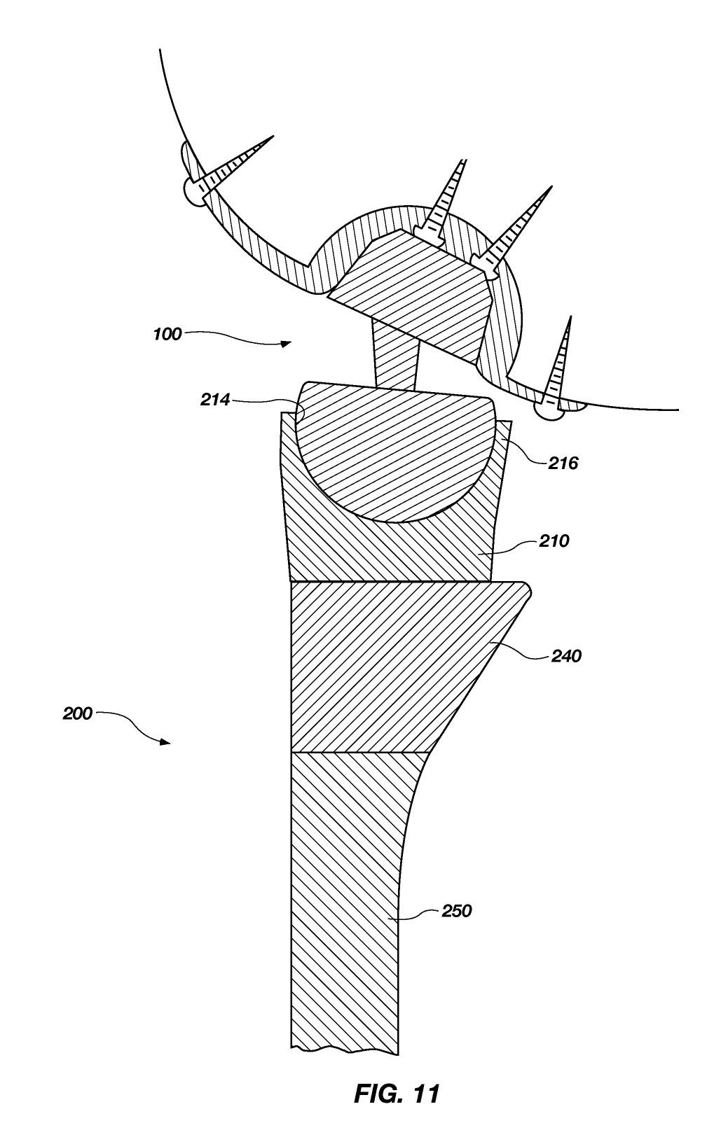

Referring to FIG. 11, the concave articulation surface portion 210 of the second component 200 may comprise a wall 216 defining the concave articulation surface 214 and further defining an overall area of said concave articulation surface 214. It will be appreciated that the wall 216 may be configured and arranged to extend around at least a portion of the convex articulation surface 147 of the insert 140 of the first component 100. The greater the surface area of the concave articulation surface 214, which may be created and defined by the wall 216, the more constrained the convex articulation surface 147 will be when the concave articulation surface 214 and the convex articulation surface 147 are matingly engaged. In other words, the higher the wall 216 the more force that is required for the convex articulation surface 147 of the head 146 to jump or move over the top of the wall 216. Thus, it will be appreciated that stability of the concave-convex articulation surface interface is increased as the height of the wall 216 increases, thereby becoming more constrained. As the wall 216 increases in height, a hemispherical capture of the head 146 and the convex articulation surface 147 may occur with respect to the concave articulation surface 214.

In a substantially non-constrained embodiment of the present disclosure, the wall 216 may not function to constrain the convex articulation surface 147 of the head 146 to a large degree. In other words, in the substantially non-constrained embodiment less than about twenty percent of the convex articulation surface 147 of the head 146, when seated in the concave articulation surface 214, is surrounded by the wall 216.

In another embodiment, the wall 216 may be formed such that it may extend around the convex articulation surface 147 of the insert 140 in a semi-constrained manner. In other words, about twenty percent to about fifty percent of the convex articulation surface 147 of the head 146, when seated in the concave articulation surface 214, is surrounded by the wall 216 (see FIG. 1).

It has been found to be advantageous that the wall 216 may extend around at least thirty percent of the convex articulation surface 147 of the insert 140. As the contact interface between the convex articulation surface 147 of the liner 140 and the concave articulation surface 214 of the concave articulation surface portion 210 increases the amount of stress at the interface decreases. Thus, there is a lower potential coefficient of friction and therefore a reduction in wear debris generation at the interface. Thus, by maximizing the surface area contact between the convex articulation surface 147 and the concave articulation surface 214, the coefficient of friction at the interface is reduced and the amount of stress at the interface is also reduced. The above advantages are made possible by the design of the present disclosure, i.e., with the convex articulation surface 147 being located on the acetabular side of the hip joint and the concave articulation surface 214 being located on the femoral side of the hip joint.

In another embodiment, the wall 216 may be formed such that it may extend around the convex articulation surface 147 of the head 146 of the insert 140 in a constrained manner. In other words, more than about fifty percent of the convex articulation surface 147 of the head 146, when seated in the concave articulation surface 214, is surrounded by the wall 216, as illustrated in FIG. 11.

One of skill in the art can readily determine the indications of when a substantially non-constrained embodiment, a semi-constrained embodiment or a fully constrained embodiment is advantageous over the others. For example, a patient who has chronic dislocation problems is a prime candidate for a fully constrained embodiment, whereas a semi-constrained or potentially a substantially non-constrained embodiment may be used for active patients.

It will be appreciated that the distal stem portion 250 may be lengthened or shortened depending upon the desired outcome. In one embodiment, the distal stem portion 250 may be at least 200 mm in length and may be bowed or curved in a manner that substantially matches the shape of the medullary canal of the patient's femur. In such instances, the modular aspects of the bi-body or tri-body stems of the present disclosure may be advantageously used to increase the bony contact between the proximal stem portion 210 and the medial calcar portion of the femur. Increased bony contact by the second component may increase the overall joint stability of the entire implant.

If a modular distal stem portion 250 or even a component comprising both the proximal body portion 240 and the distal stem portion 250 as a single component (but that is modular with respect to the concave articulation surface portion 210), has been implanted in a patient's body and should the patient require a revision surgery, then the second component 200 can be utilized in a traditional hip arthroplasty. In other words, the modular concave articulation surface portion 210 can be removed from the patient leaving the distal stem portion 250, or the proximal body portion 240 and the distal stem portion 250, in place in the patient's femoral canal. Due to its modularity, the concave articulation surface portion 210 can be replaced with a traditional femoral neck and head. In such a case, only the acetabular component, i.e., the first component 100, will have to be completely removed, thereby saving valuable operating time and increasing the efficacy of the second component 200 because the distal stem component 250 does not have to be removed or replaced.

It will be appreciated that the structure and apparatus disclosed herein regarding the second component 200 is merely one example of a means for articulating with the convex articulation surface 147 of the insert 140 in a semi-constrained manner, and it should be appreciated that any structure, apparatus or system for articulating with the convex articulation surface 147 of the insert 140 in a semi-constrained manner, which performs functions the same as, or equivalent to, those disclosed herein are intended to fall within the scope of a means for articulating with the convex articulation surface 147 of the insert 140 in a semi-constrained manner, including those structures, apparatus or systems for articulating with the convex articulation surface 147 of the insert 140 in a semi-constrained manner, which are presently known, or which may become available in the future. Anything which functions the same as, or equivalently to, a means for articulating with the convex articulation surface 147 of the insert 140 in a semi-constrained manner falls within the scope of this element.

It will be appreciated that the structure and apparatus disclosed herein is merely one example of a means for indexing one modular component with respect to another component, and it should be appreciated that any structure, apparatus or system for indexing one modular component with respect to another component, which performs functions the same as, or equivalent to, those disclosed herein are intended to fall within the scope of a means for indexing one modular component with respect to another component, including those structures, apparatus or systems for indexing one modular component with respect to another component, which are presently known, or which may become available in the future. Anything which functions the same as, or equivalently to, a means for indexing one modular component with respect to another component falls within the scope of this element.

It will be appreciated that the structure and apparatus disclosed herein is merely one example of a means for interlocking one modular component with respect to another component, and it should be appreciated that any structure, apparatus or system for interlocking one modular component with respect to another component, which performs functions the same as, or equivalent to, those disclosed herein are intended to fall within the scope of a means for interlocking one modular component with respect to another component, including those structures, apparatus or systems for interlocking one modular component with respect to another component, which are presently known, or which may become available in the future. Anything which functions the same as, or equivalently to, a means for interlocking one modular component with respect to another component falls within the scope of this element.

In accordance with the features and combinations described above, a useful method of restoring joint mechanics in a hip joint, may comprise the steps of:

(a) providing an acetabular or first component 100 comprising a convex articulation surface 147 and a femoral or second component 200 comprising a concave articulation surface 214;

(b) implanting the acetabular or first component 100 in a surgically prepared acetabulum of a patient such that the convex articulation surface 147 extends from the patient's acetabulum; and

(c) implanting the femoral or second component 200 in a surgically prepared proximal femur, such that the concave articulation surface 214 extends from the proximal femur in an orientation to receive the convex articulation surface 147 of the acetabular or first component 100.

Those having ordinary skill in the relevant art will appreciate the advantages provide by the features of the present disclosure. For example, it is a potential feature of the present disclosure to restore the normal or natural joint mechanics using the components disclosed herein, or in other words, target restoration of the natural joint mechanics in, for example, a hip joint. Another potential feature of the present disclosure is to stabilize the joint by utilizing the device disclosed herein. It is yet another potential feature of the present disclosure to reduce stress and strains at the bearing or articulating interface between the first component 100 and the second component 200, to thereby reduce wear debris. Another potential feature includes providing modular, indexable components to aid in correcting version, offset in a joint.

In the foregoing Detailed Description of the Disclosure, various features of the present disclosure are grouped together in a single embodiment for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description of the Disclosure by this reference, with each claim standing on its own as a separate embodiment of the present disclosure.

It is to be understood that the above-described arrangements are only illustrative of the application of the principles of the present disclosure. Numerous modifications and alternative arrangements may be devised by those skilled in the art without departing from the spirit and scope of the present disclosure and the appended claims are intended to cover such modifications and arrangements. Thus, while the present disclosure has been shown in the drawings and described above with particularity and detail, it will be apparent to those of ordinary skill in the art that numerous modifications, including, but not limited to, variations in number, size, materials, shape, form, function and manner of operation, assembly and use may be made without departing from the principles and concepts set forth herein.

What is claimed is:

1. An orthopedic device comprising:
a first component attachable to an acetabulum of a patient;
a second component attachable to a proximal femur of the patient;
wherein the first component comprises a shell that is directly attachable to the acetabulum of the patient and an insert that is attachable to the shell;
wherein the insert has a convex articulation surface and the second component has a concave articulation surface that engages the convex articulation surface of the insert of the first component to form an artificial joint.

2. The orthopedic device of claim 1, wherein the insert includes a tapered outer surface and the shell includes a cavity defined at least in part by a tapered wall for matingly engaging the tapered outer surface of the insert in a self-locking friction fit.

3. The orthopedic device of claim 2, wherein the tapered outer surface of the insert and the tapered wall of the shell comprise an angle that is within a range of self-locking taper angles.

4. The orthopedic device of claim 2, wherein the shell is attached to the acetabulum using bone cement.

5. The orthopedic device of claim 2, wherein the shell is attached to the acetabulum via a press-fit between the shell and bone of the acetabulum itself.

6. The orthopedic device of claim 1, wherein the shell is substantially semi-spherical in shape.

7. The orthopedic device of claim 1, wherein the shell comprises a first body and at least one flange that extends from the first body.

8. The orthopedic device of claim 7, wherein the at least one flange comprises a through hole such that a fastener is extendable into the through hole and into the surrounding bone.

9. The orthopedic device of claim 1, wherein the shell comprises a cavity defined by a wall and wherein at least one through hole is formed within the wall of the cavity, such that a fastener is extendable into the through hole and into the surrounding bone.

10. The orthopedic device of claim 1, wherein the shell comprises a cavity and the insert further comprises a body, a neck and a head.

11. The orthopedic device of claim 10, wherein the insert comprises at least one modular connection with respect to the body and the neck, and between the neck and the head.

12. The orthopedic device of claim 11, wherein the at least one modular connection is a self-locking taper interlock.

13. The orthopedic device of claim 10, wherein the head is affixed to the neck and the neck is affixed to the body and extends outwardly with respect to said body, and wherein said body of the insert is attachable to the cavity of the shell.

14. The orthopedic device of claim 11, wherein the neck is modular with respect to the body and comprises an outer tapered portion and said body has a recess defined by a tapered sidewall, wherein the outer tapered portion of the neck engages the tapered sidewall of the recess of the body in a tapered friction fit.

15. The orthopedic device of claim 14, wherein the outer tapered portion of the neck comprises a plurality of first teeth substantially surrounding a perimeter of the outer tapered portion, wherein the tapered sidewall of the recess in the body comprises a corresponding plurality of second teeth for engaging the first teeth, such that the neck is indexable in a plurality of differing orientations with respect to the body.

16. The orthopedic device of claim 10, wherein the insert comprises a central body axis and a central neck axis, wherein the central neck axis is offset at an angle with respect to the central body axis.

17. The orthopedic device of claim 16, wherein the angle between axes is within a range of about ten degrees to about sixty degrees.

18. The orthopedic device of claim 17, wherein the angle between axes is within a range of about twenty degrees to about fifty degrees.

19. The orthopedic device of claim 18, wherein the angle between axes is about thirty-five degrees.

20. The orthopedic device of claim 10, wherein the body comprises a first base portion and a recess defined in the first base portion by a tapered sidewall, wherein the neck comprises a second base portion and a tapered outer portion extending from the second base portion for engaging the tapered sidewall in a friction fit, wherein the neck further comprises a support at the second base portion creating an angular offset of the neck with respect to the first base portion of the body when the neck is attached to the body.

21. The orthopedic device of claim 20, wherein the neck comprises a plurality of first teeth substantially surrounding a perimeter of the outer tapered portion, wherein the tapered sidewall of the recess in the body comprises a corresponding plurality of second teeth for engaging the first teeth, such that the neck is indexable in a plurality of differing orientations with respect to the body, and wherein the angular offset of the neck creates a differing version angle as the neck is oriented in each of the plurality of differing orientations.

22. The orthopedic device of claim 20, wherein the support is shaped as a wedge and the angular offset is within a range of angles of about three degrees to about twenty degrees.

23. The orthopedic device of claim 22, wherein the angular offset is within a range of angles of about six degrees to about fifteen degrees.

24. The orthopedic device of claim 1, wherein the concave articulation surface is part of a concave articulation surface portion, and wherein the second component is a femoral stem component that further comprises a distal stem portion and a proximal body portion.

25. The orthopedic device of claim 24, wherein the second component is a monoblock stem.

26. The orthopedic device of claim 24, wherein the second component is a bi-body stem having two modular pieces chosen from the group consisting of the concave articulation surface portion, the proximal body portion and the distal stem portion.

27. The orthopedic device of claim 24, wherein the second component is a tri-body stem, wherein the concave articulation surface portion, the proximal body portion and the distal stem portion are modular pieces with respect to each other.

28. The orthopedic device of claim 24, wherein the second component is a modular stem and has at least one modular junction formed between at least two components selected from the group consisting of the concave articulation surface portion, the proximal body portion and the distal stem portion.

29. The orthopedic device of claim 28, wherein the at least two components are joined at the modular junction by a first tapered portion formed as part of one of the modular components engaging a sidewall defining a recess in another one of the modular components in a friction fit.

30. The orthopedic device of claim 24, wherein the concave articulation surface portion comprises a wall defining the concave articulation surface, wherein the wall extends around at least a portion of the convex articulation surface of the insert.

31. The orthopedic device of claim 30, wherein the wall extends around the convex articulation surface of the insert in a semi-constrained manner.

32. The orthopedic device of claim 30, wherein the wall extends around the convex articulation surface of the insert in a constrained manner.

33. The orthopedic device of claim 30, wherein the wall extends around at least thirty percent of the convex articulation surface of the insert.

34. The orthopedic device of claim 28, wherein the distal stem portion is at least 200 mm in length and is bowed in a manner to substantially match the shape of the medullary canal.

35. The orthopedic device of claim 1, wherein the concave articulation surface is a modular attachment piece that is securable to a concave articulation surface portion.

36. The orthopedic device of claim 35, wherein the modular attachment piece that is the concave articulation surface is one of a plurality of modular attachment pieces each having a different thickness than the others, wherein each of the plurality of modular attachment pieces provides a surgeon with an ability to adjust the joint mechanics by utilizing a particular modular attachment piece having a particular thickness.

37. The orthopedic device of claim 36, wherein each of the modular attachment pieces is lockable to the concave articulation surface portion.

38. The orthopedic device of claim 35, wherein the concave articulation surface and the concave articulation surface portion are manufactured from a material selected from a group consisting of ceramic, metal or diamond.

39. The orthopedic device of claim 35, wherein the concave articulation surface is manufactured from a material selected from a group consisting of ceramic, metal or diamond, and the concave articulation surface portion is manufactured from a polymeric material.

40. An orthopedic device comprising:
a first component attachable to an acetabulum of a patient comprising a convex articulation surface; and
a second component attachable to a proximal femur of the patient comprising a concave articulation surface portion; wherein the second component comprises a plurality of modular attachment pieces each having a concave articulation surface and each having a different thickness, wherein the concave articulation surface of each modular attachment piece is engageable with the convex articulation surface of the first component after implantation in a patient's body.

41. The orthopedic device of claim 40, wherein each of the plurality of modular attachment pieces is securable to the concave articulation surface portion of the second component.

42. The orthopedic device of claim 40, wherein the first component comprises a shell that is directly attachable to the acetabulum of the patient and an insert that is attachable to the shell, wherein the insert comprises the convex articulation surface.

43. The orthopedic device of claim 40, wherein the second component is a femoral stem component that further comprises a distal stem portion and a proximal body portion.

44. The orthopedic device of claim 43, wherein the concave articulation surface portion, the proximal body portion and the distal stem portion comprise a monoblock stem, such that the only modular aspect of the second component is the modular attachment pieces.

45. The orthopedic device of claim 43, wherein the second component is a bi-body stem having two modular pieces chosen from the group consisting of the concave articulation surface portion, the proximal body portion and the distal stem portion.

46. The orthopedic device of claim 43, wherein the second component is a tri-body stem, wherein the concave articulation surface portion, the proximal body portion and the distal stem portion are modular pieces with respect to each other.

47. The orthopedic device of claim 43, wherein the second component is a modular stem and has at least one modular junction formed between at least two components selected from the group consisting of the concave articulation surface portion, the proximal body portion and the distal stem portion.

48. The orthopedic device of claim 47, wherein the at least two components are joined at the modular junction by a first tapered portion formed as part of one of the modular components engaging a sidewall defining a recess in another one of the modular components in a friction fit.

49. The orthopedic device of claim 43, wherein the concave articulation surface portion comprises a wall defining the concave articulation surface, wherein the wall extends around at least a portion of the convex articulation surface of the insert.

50. The orthopedic device of claim 49, wherein the wall extends around the convex articulation surface of the insert in a semi-constrained manner.

51. The orthopedic device of claim 49, wherein the wall extends around the convex articulation surface of the insert in a constrained manner.

52. The orthopedic device of claim 49, wherein the wall extends around at least thirty percent of the convex articulation surface of the insert.

53. The orthopedic device of claim 47, wherein the distal stem portion is at least 200 mm in length and is bowed in a manner to substantially match the shape of the medullary canal.

54. The orthopedic device of claim 40, wherein each of the modular attachment pieces is securable and lockable to the concave articulation surface portion.

55. The orthopedic device of claim 40, wherein each of the plurality of modular attachment pieces provides a surgeon with an ability to adjust the joint mechanics by utilizing a particular modular attachment piece having a particular thickness.

56. The orthopedic device of claim 40, wherein each of the modular attachment pieces comprises a different surface area than the other modular attachment pieces, thereby creating a constrained, semi-constrained or non constrained concave articulation surface.

57. The orthopedic device of claim 40, wherein at least one of the concave articulation surfaces of at least one of the plurality of modular attachment pieces and the concave articulation surface portion are manufactured from a material selected from a group consisting of ceramic, metal or diamond.

58. The orthopedic device of claim 40, wherein at least one of the concave articulation surfaces is manufactured from a material selected from a group consisting of ceramic, metal or diamond, and the concave articulation surface portion is manufactured from a polymeric material.

59. An orthopedic device comprising:
a femoral component configured and arranged to be attached to a patient's proximal femur comprising a concave articulation surface;
a modular acetabular component configured and arranged to be attached to the patient's acetabulum, wherein the modular acetabular component comprises an acetabular shell and an insert;
wherein the acetabular shell includes a cavity defined by a tapering sidewall;
wherein the insert comprises a convex articulation surface and a body that is defined by a tapered outer surface;
wherein the tapered outer surface of the body of the insert matingly engages the tapering sidewall of the cavity to secure the insert to the shell via a self locking tapered fit.

60. The orthopedic device of claim 59, wherein the insert further comprises a head that comprises the convex articulation surface and a neck that extends between the body and the head.

61. The orthopedic device of claim 60, wherein the insert comprises at least one modular connection with respect to the body and the neck, and between the neck and the head.

62. The orthopedic device of claim 61, wherein the at least one modular connection is a self-locking taper interlock.

63. The orthopedic device of claim 60, wherein the head of the insert is affixed to the neck of the insert and said neck is affixed to and extends from the body of the insert.

64. The orthopedic device of claim 61, wherein the neck of the insert is modular with respect to the body of the insert and comprises an outer tapered portion and said body has a recess defined by a tapered sidewall, wherein the outer tapered portion of the neck engages the tapered sidewall of the recess of the body in a tapered friction fit.

65. The orthopedic device of claim 64, wherein the outer tapered portion of the neck comprises a plurality of first teeth substantially surrounding a perimeter of the outer tapered portion, wherein the tapered sidewall of the recess in the body comprises a corresponding plurality of second teeth for engaging the first teeth, such that the neck is indexable in a plurality of differing orientations with respect to the body.

66. The orthopedic device of claim 60, wherein the insert comprises a central body axis and a central neck axis, wherein the central neck axis is offset at an angle with respect to the central body axis.

67. The orthopedic device of claim 66, wherein the angle between axes is within a range of about ten degrees to about sixty degrees.

68. The orthopedic device of claim 67, wherein the angle between axes is within a range of about twenty degrees to about fifty degrees.

69. The orthopedic device of claim 68, wherein the angle between axes is about thirty-five degrees.

70. The orthopedic device of claim 60, wherein the body of the insert comprises a first base portion and a recess defined in the first base portion by a tapered sidewall, wherein the neck of the insert comprises a second base portion and a tapered outer portion extending from the second base portion for engaging the tapered sidewall in a friction fit, wherein the neck further comprises a support at the second base portion creating an angular offset of the neck with respect to the first base portion of the body when the neck is attached to the body.

71. The orthopedic device of claim 70, wherein the neck comprises a plurality of first teeth substantially surrounding a perimeter of the outer tapered portion, wherein the tapered sidewall of the recess in the body comprises a corresponding plurality of second teeth for engaging the first teeth, such that the neck is indexable in a plurality of differing orientations with respect to the body, and wherein the angular offset of the neck creates a differing version angle as the neck is oriented in each of the plurality of differing orientations.

72. The orthopedic device of claim 70, wherein the support is shaped as a wedge and the angular offset is within a range of angles of about three degrees to about twenty degrees.

73. The orthopedic device of claim 72, wherein the angular offset is within a range of angles of about six degrees to about fifteen degrees.

74. The orthopedic device of claim 59, wherein the shell is attached to a patient's acetabulum using bone cement.

75. The orthopedic device of claim 59, wherein the shell is attached to a patient's acetabulum via a press-fit between the shell and bone of the acetabulum itself.

76. The orthopedic device of claim 59, wherein the shell is substantially semi-spherical in shape.

77. The orthopedic device of claim 59, wherein the shell comprises a first body and at least one flange that extends from the first body.

78. The orthopedic device of claim 78, wherein the at least one flange comprises a through hole such that a fastener is extendable into the through hole and into the surrounding bone.

79. The orthopedic device of claim 59, wherein the tapering sidewall defining the cavity of the shell comprises at least one through hole, such that a fastener is extendable into the through hole and into the surrounding bone.

80. An orthopedic device comprising:
a first component that is directly attachable to an acetabulum of a patient and comprising a convex articulation surface; and
a second component that is directly attachable to a proximal femur of the patient;
wherein the second component comprises a concave articulation surface, a proximal body portion and a distal stem portion;
wherein at least one modular junction is formed in the second component between the concave articulation surface and the proximal body portion, and between the proximal body portion and the distal stem portion.

81. The orthopedic device of claim 80, wherein the second component is a bi-body stem having two modular pieces chosen from the group consisting of the proximal articulation surface, the proximal body portion and the distal stem portion.

82. The orthopedic device of claim 80, wherein the second component is a tri-body stem, wherein the proximal articulation surface, the proximal body portion and the distal stem portion are modular pieces with respect to each other.

83. The orthopedic device of claim 80, wherein the proximal articulation surface, the proximal body portion, and the distal stem portion are modular components, and wherein the at least one modular junction is formed by a first tapered portion formed as part of one of the modular components engaging a sidewall defining a recess in another one of the modular components in a friction fit.

84. The orthopedic device of claim 80, wherein the proximal articulation surface comprises a wall defining the concave articulation surface, wherein the wall extends around at least a portion of the convex articulation surface of the first component.

85. The orthopedic device of claim 84, wherein the wall extends around the convex articulation surface of the first component in a semi-constrained manner.

86. The orthopedic device of claim 84, wherein the wall extends around the convex articulation surface of the first component in a constrained manner.

87. The orthopedic device of claim 84, wherein the wall extends around at least thirty percent of the convex articulation surface of the first component.

88. The orthopedic device of claim 80, wherein the distal stem portion is at least 200 mm in length and is bowed in a manner to substantially match the shape of a patient's medullary canal.

89. An orthopedic device comprising:
a first component that is directly attachable to an acetabulum of a patient and comprising a shell and an insert, wherein the shell comprises a cavity and the insert is attachable within the cavity of the shell, and wherein the insert comprises a convex articulation surface; and
a second component that is directly attachable to a proximal femur of the patient;
wherein the second component comprises a means for articulating with the convex articulation surface of the insert in a semi-constrained manner.

90. An orthopedic device comprising:
an acetabular component that is directly attachable to an acetabulum of a patient comprising a convex articulation surface; and
a femoral component that is directly attachable to a proximal femur of the patient comprising a concave articulation surface that engages the convex articulation surface of the acetabular component upon surgical implantation of the device within the patient's body;
wherein the concave articulation surface is defined by a wall that surrounds at least thirty percent of the convex articulation surface of the acetabular component.

91. The orthopedic device of claim 90, wherein the wall of the concave articulation surface surrounds the convex articulation surface of the acetabular component in a constrained manner.

92. The orthopedic device of claim 90, wherein the wall of the concave articulation surface surrounds the convex articulation surface of the acetabular component in a semi-constrained manner.

93. An orthopedic device comprising:
a first component attachable to an acetabulum of a patient comprising a convex articulation surface;
a second component attachable to a proximal femur of the patient comprising a concave articulation surface;
wherein the first component comprises a shell that is directly attachable to the acetabulum of the patient and an insert comprising a body and a neck that is attachable to the shell;
wherein the insert further comprises a central body axis and a central neck axis that is offset at an angle with respect to the central body axis.

94. The orthopedic device of claim 93, wherein the angle between axes is within a range of about ten degrees to about sixty degrees.

95. The orthopedic device of claim 94, wherein the angle between axes is within a range of about twenty degrees to about fifty degrees.

96. The orthopedic device of claim 95, wherein the angle between axes is about thirty-five degrees.

97. The orthopedic device of claim 93, wherein the insert comprises a head and the convex articulation surface that engages the concave articulation surface of the second component to form an artificial joint.

98. A method of restoring joint mechanics in a hip joint, comprising the steps of:
(a) providing an acetabular component comprising a convex articulation surface and a femoral component comprising a concave articulation surface;
(b) implanting the acetabular component in a surgically prepared acetabulum of a patient such that the convex articulation surface extends from the patient's acetabulum; and
(c) implanting the femoral component in a surgically prepared proximal femur, such that the concave articulation surface extends from the proximal femur in an orientation to receive the convex articulation surface of the acetabular component.

99. An orthopedic device comprising:
a first component attachable to an acetabulum of a patient;
a second component attachable to a proximal femur of the patient;
wherein the first component comprises a shell that is directly attachable to the acetabulum of the patient and an insert that is attachable to the shell;
wherein the insert has a convex articulation surface;
wherein the second component has a concave articulation surface that engages the convex articulation surface of the insert to form an artificial joint;
wherein the insert includes a tapered outer surface and the shell includes a cavity defined at least in part by a tapered wall for matingly engaging the tapered outer surface of the insert in a self-locking friction fit;
wherein the tapered outer surface of the insert and the tapered wall of the shell comprise an angle that is within a range of self-locking taper angles;
wherein the shell is substantially semi-spherical in shape;
wherein the shell comprises a first body and at least one flange that extends from the first body;
wherein the at least one flange comprises a first through hole such that a fastener is extendable into the through hole and into the surrounding bone;
wherein at least one second through hole is formed within the wall of the cavity of the shell, such that a fastener is extendable through the second through hole and into the surrounding bone;
wherein the insert further comprises a second body, a neck and a head;
wherein the insert comprises at least one modular connection between the second body and the neck or between the neck and the head, wherein the at least one modular connection is a self-locking taper interlock;
wherein the head of the insert is attached to the neck and the neck is attached to the second body and extends outwardly with respect to said second body;
wherein the insert comprises a central body axis and a central neck axis, wherein the central neck axis is offset at an angle with respect to the central body axis;
wherein the angle between axes is within a range of about ten degrees to about sixty degrees;
wherein the concave articulation surface is part of a concave articulation surface portion;
wherein the second component is a femoral stem component that further comprises a distal stem portion and a proximal body portion; and
wherein the concave articulation surface portion comprises a wall defining the concave articulation surface, wherein the wall extends around at least a portion of the convex articulation surface of the insert.

100. The orthopedic device of claim 99, wherein the concave articulation surface is a modular attachment piece that is securable to the concave articulation surface portion.

101. The orthopedic device of claim 100, wherein the modular attachment piece that is the concave articulation surface is one of a plurality of modular attachment pieces each having a different thickness than the others, wherein each of the plurality of modular attachment pieces provides a surgeon with an ability to adjust the joint mechanics by utilizing a particular modular attachment piece having a particular thickness.

102. The orthopedic device of claim 101, wherein each of the modular attachment pieces is lockable to the concave articulation surface portion.

103. The orthopedic device of claim 100, wherein the concave articulation surface and the concave articulation surface portion are manufactured from a material selected from a group consisting of ceramic, metal or diamond.

104. The orthopedic device of claim 100, wherein the concave articulation surface is manufactured from a material selected from a group consisting of ceramic, metal or diamond, and the concave articulation surface portion is manufactured from a polymeric material.

105. The orthopedic device of claim 99, wherein the neck of the insert is modular with respect to the second body and comprises an outer tapered portion and said second body has a recess defined by a tapered sidewall, wherein the outer tapered portion of the neck engages the tapered sidewall of the recess of the second body in a tapered friction fit.

106. The orthopedic device of claim 105, wherein the outer tapered portion of the neck comprises a plurality of first teeth substantially surrounding a perimeter of the outer tapered portion, wherein the tapered sidewall of the recess in the second body comprises a corresponding plurality of second teeth for engaging the first teeth, such that the neck is indexable in a plurality of differing orientations with respect to the second body.

107. The orthopedic device of claim 99, wherein the angle between axes is within a range of about twenty degrees to about fifty degrees.

108. The orthopedic device of claim 107, wherein the angle between axes is about thirty-five degrees.

109. The orthopedic device of claim 99, wherein the second body of the insert comprises a first base portion and a recess defined in the first base portion by a tapered sidewall, wherein the neck comprises a second base portion and a tapered outer portion extending from the second base portion for engaging the tapered sidewall in a friction fit, wherein the neck further comprises a support at the second base portion creating an angular offset of the neck with respect to the first base portion of the second body when the neck is attached to the second body.

110. The orthopedic device of claim 109, wherein the neck comprises a plurality of first teeth substantially surrounding a perimeter of the outer tapered portion, wherein the tapered sidewall of the recess in the second body comprises a corresponding plurality of second teeth for engaging the first teeth, such that the neck is indexable in a plurality of differing orientations with respect to the second body, and wherein the angular offset of the neck creates a differing version angle as the neck is oriented in each of the plurality of differing orientations.

111. The orthopedic device of claim 109, wherein the support is shaped as a wedge and the angular offset is within a range of angles of about three degrees to about twenty degrees.

112. The orthopedic device of claim 111, wherein the angular offset is within a range of angles of about six degrees to about fifteen degrees.

113. The orthopedic device of claim 99, wherein the second component is a monoblock stem.

114. The orthopedic device of claim 99, wherein the second component is a bi-body stem having two modular pieces chosen from the group consisting of the concave articulation surface portion, the proximal body portion and the distal stem portion.

115. The orthopedic device of claim 99, wherein the second component is a tri-body stem, wherein the concave articulation surface portion, the proximal body portion and the distal stem portion are modular pieces with respect to each other.

116. The orthopedic device of claim 99, wherein the second component is a modular stem and has at least one modular junction formed between at least two components selected from the group consisting of the concave articulation surface portion, the proximal body portion and the distal stem portion.

117. The orthopedic device of claim 99, wherein the at least two components are joined at the modular junction by a first tapered portion formed as part of one of the modular components engaging a sidewall defining a recess in another one of the modular components in a friction fit.

118. The orthopedic device of claim 99, wherein the wall defining the concave articulation surface extends around the convex articulation surface of the insert in a semi-constrained manner.

119. The orthopedic device of claim 99, wherein the wall defining the concave articulation surface extends around the convex articulation surface of the insert in a constrained manner.

120. The orthopedic device of claim 99, wherein the wall defining the concave articulation surface extends around at least thirty percent of the convex articulation surface of the insert.

121. The orthopedic device of claim 99, wherein the distal stem portion is at least 200 mm in length and is bowed in a manner to substantially match the shape of the medullary canal.

122. An orthopedic device comprising:
a first component attachable to an acetabulum of a patient comprising a shell and an insert that is attachable to the shell; and
a modular attachment piece that is attachable to a femoral stem component of an artificial hip implant, wherein the modular attachment piece comprises a concave articulation surface;
wherein the insert comprises a convex articulation surface that engages the concave articulation surface to form an artificial joint.

123. The orthopedic device of claim 122, wherein the modular attachment piece comprises a cup with a substantially semi-spherical cavity formed therein, wherein the cavity is defined by the concave articulation surface.

124. The orthopedic device of claim 123, wherein the modular attachment piece comprises a support extending from the cup, wherein a recess is formed within the support and is defined by a tapered sidewall for matingly engaging a tapered end portion of a neck portion of the femoral component in a friction fit.

125. The orthopedic device of claim 123, wherein the cup of the modular attachment piece is configured to constrain the convex articulation surface and is attachable to a femoral stem component that is existing and implanted within the femur of the patient during a revision surgery.

126. The orthopedic device of claim 122, wherein the device comprises an adapter that is attachable to the modular attachment piece, wherein a recess is formed within the adapter and is defined by a tapered sidewall for matingly engaging a tapered end portion of a neck portion of the femoral component in a friction fit.

127. The orthopedic device of claim 126, wherein the concave articulation surface of the modular attachment piece does not substantially constrain the convex articulation surface.

128. The orthopedic device of claim 1, wherein the second component comprises a modular attachment piece that is attachable to a femoral stem component, wherein the modular attachment piece comprises the concave articulation surface that engages the convex articulation surface of the first component.

129. The orthopedic device of claim 128, wherein the modular attachment piece comprises a cup with a substantially semi-spherical cavity formed therein, wherein the cavity is defined by the concave articulation surface.

130. The orthopedic device of claim 129, wherein the modular attachment piece comprises a support extending from the cup, wherein a recess is formed within the support and is defined by a tapered sidewall for matingly engaging a tapered end portion of a neck portion of the femoral component in a friction fit.

131. The orthopedic device of claim 129, wherein the cup of the modular attachment piece is configured to constrain the convex articulation surface and is attachable to a femoral stem component that is existing and implanted within the femur of the patient during a revision surgery.

132. The orthopedic device of claim 128, wherein the device comprises an adapter that is attachable to the modular attachment piece, wherein a recess is formed within the adapter and is defined by a tapered sidewall for matingly engaging a tapered end portion of a neck portion of the femoral component in a friction fit.

133. The orthopedic device of claim 132, wherein the concave articulation surface of the modular attachment piece does not substantially constrain the convex articulation surface.
